(19) United States
(12) Patent Application Publication
(10) Pub. No.: US 2009/0005342 A1
(43) Pub. Date: Jan. 1, 2009

(54) Composition Having Blood Pressure Reducing and/or Elevation Suppressing Effect and Food and Drink Containing the Same

(76) Inventors: Izumi Takao, Mie (JP); Ichiro Asano, Tokyo (JP); Shigeyoshi Fujii, Mie (JP); Masayuki Kaneko, Tokyo (JP); John R. Nielson, Melbourne (AU); Daniel G. Steffen, Long Beach Twp, NJ (US); Thomas Hatzold, Munchen (DE)

Correspondence Address: FITCH EVEN TABIN & FLANNERY, 120 S. LASALLE STREET, SUITE 1600, CHICAGO, IL, 60603-3406, US

(21) Appl. No.: 12/178,296

(22) Filed: Jul. 23, 2008

Related U.S. Application Data
(63) Continuation-in-part of application No. PCT/JP2006/301025, filed on Jan. 24, 2006.

(51) Int. Cl. A61K 31/702; C08B 37/00; A61P 9/12

(52) U.S. Cl. 514/54; 536/123.1

(57) ABSTRACT

The present invention regards the composition having a blood pressure reducing and/or elevation suppressing effect comprising a mannose-based oligosaccharide mass comprising 1 to 10 molecules of monosaccharides linked together, said molecules mainly comprising mannose. It is the object of the present invention to provide an economical and simple food and drink which has an excellent blood pressure reducing and/or elevation suppressing effect without changing ordinary eating habits much, and a composition ingestible with a food and drink which effectively uses waste resources such as coffee extraction residues.

### CROSS-REFERENCE TO RELATED APPLICATIONS

This is a Continuation-In-Part (CIP) application of International application PCT/JP2006/301025 filed on Jan. 24, 2006, which is hereby entirely incorporated herein by reference.

### FIELD OF THE INVENTION

The present invention relates to a composition having a blood pressure reducing and/or elevation suppressing effect in mammals, especially humans. This composition comprises, as the main component, oligosaccharides composed of constituent monosaccharides mainly comprising mannose in an oral ingestible form particularly a food and drink. The present invention also provides a method for preventing, treating, ameroliating, controlling, or otherwise effecting high blood pressure or hypertension in a human. Further, the present invention relates to effective utilization of unused resources.

### BACKGROUND

Almost all of coffee extracted residues have previously been burned up, or treated as an industrial waste. Some coffee extracted residues have more recently become used as a compost raw material or an active carbon raw material, which is, however, less than sufficient in view of advanced utilization of unused resources; therefore, establishing further methods for advanced utilization of coffee extracted residues represents an important challenge.

Lifestyle-related diseases such as diabetes, hypertension, and hyperlipemia, which are rapidly increasing in recent years, are considered to be closely related to dietary life, and emphasis is placed on the prevention thereof by improved dietary life. High blood pressure or hypertension is a condition which occurs in about 50 percent of elderly people (60 years and above) in Japan. If high blood pressure is left untreated, the risk of stroke, cardiac infarction or cardiac hypertrophy caused by sclerosis of the arteries, and related conditions is increased. Therapeutic drugs for treating high blood pressure include, for example, diuretic agents, sympathetic blocking agents, vasodilatory agents, and so on. Although such drugs generally can be effective in controlling high blood pressure if taken in a consistent manner, side effects (e.g., edema, polyuria, palpitation, and the like) can occur. Moreover, prevention of the development of hypertension is preferred to treatment of the condition after it develops. And where possible, it is generally preferred to use non-drug treatment methods as opposed to drug methods.

For these reason, non-drug preventative and treatment methods such as improvements in lifestyle, diet, participating in a regular exercise regime, and limiting the intake of alcohol and tobacco are recommended. In particular, improvement in everyday dietary life is emphasized because an improvement of diet alone may prevent high blood pressure, suppress the progression of the condition, and even prevent the development of the condition in the first instance. Therefore, efforts have been made to identify substances in food that are beneficial in preventing high blood pressure, and the mechanism of action of these substances is studied for example, in JP 2002-80354 A and JP 2002-272420 A. Often, however, foods or drinks to which such substances have been added have undesirable taste or other organoleptic properties. The applicants of the present application have earlier found that a mannose-based oligosaccharide mass comprising 1 to 10 molecules of monosaccharides linked together, said molecules mainly comprising mannose and a small quantity of other substances obtained from food comprising mannan such as hydrolysates of coffee extraction residues have controlling intestinal functions accompanied by growth of bifidobacteria or reducing serum lipid function.

### SUMMARY

Now such mannose-based oligosaccharide masses have been found to have a blood pressure reducing and/or elevation suppressing effect. The present invention provides a simple, economical, and safe vegetable derived composition which can be used in foods and drinks and which is effective in reducing and lowering blood pressure and/or having an elevation suppressing effect without significantly effecting the organleptic properties of the foods and drinks. Importantly, the present compositions and methods do not require significant modification of dietary lifestyles since the composition can be included in foods and drinks normally consumed. Thus, compliance issues may not be as big a problem as in methods requiring specific drugs to be taken on a regular (generally daily) basis.

As a result of serious investigations conducted by the present inventors in order to solve the aforementioned problems, it is found that food materials, containing a large amount of mannan, mainly from hydrolysates of coffee extraction residues, a mannooligosaccharide mass having a degree of polymerization of 1 to 10 and having a reduced content of monosaccharide residues other than a mannose residue in the chain of oligosaccharide molecules, that is, a mannose-based oligosaccharide mass comprising 1 to 10 molecules of monosaccharides linked together, said molecules comprising mannose, such as glucose and galactose, has a blood pressure reducing and/or elevation suppressing effect. Furthermore, it is found that by obtaining noncolored acid-free mannooligosaccharide mass having a degree of polymerization of 1 to 10 and having a reduced amount of monosaccharide residues other than mannose residues in the chain of oligosaccharide molecules, the range of its application to food and drinks can be remarkably broadened.

Accordingly, embodiments of the present invention include, but are not limited to, the following numbered items.

1. A composition having a blood pressure reducing and/or elevation suppressing effect which comprises a mannose-based oligosaccharide mass comprising 1 to 10 molecules of monosaccharides linked together, said molecules mainly comprising mannose.

2. The composition having a blood pressure reducing and/or elevation suppressing effect of item 1, wherein said mannose-based oligosaccharide mass comprising 1 to 10 molecules of mannose linked together.

3. The composition having a blood pressure reducing and/or elevation suppressing effect of item 1, wherein said molecules comprising mannose and at least one monosaccharide selected from the group consisting of glucose, galactose and fructose.

4. The composition having a blood pressure reducing and/or elevation suppressing effect of item 1, wherein the level of mannose-based oligosaccharide mass is not less than 60 percent based on the total solid content in the composition.

5. The composition having a blood pressure reducing and/or elevation suppressing effect of any one of items 1 to 4, wherein the level of mannose residue in the mannose-based oligosaccharide mass is greater than 70 percent based on the total number of residues.

6. The composition having a blood pressure reducing and/or elevation suppressing effect of any one of items 1 to 5, wherein said mannose-based oligosaccharide mass comprising 2 to 6 molecules of mannose units in the oligosaccharide molecules.

7. The composition having a blood pressure reducing and/or elevation suppressing effect of any one of items 1 to 6, wherein said mannose-based oligosaccharide mass is obtained by the hydrolysis treatment of mannan.

8. The composition having a blood pressure reducing and/or elevation suppressing effect of item 7, wherein said mannan is obtained from coffee beans and/or coffee extraction residues.

9. The composition having a blood pressure reducing and/or elevation suppressing effect of any one of items 1 to 8, wherein said mannose-based oligosaccharide mass is β-1,4-mannooligosaccharide mass.

10. An oral ingestible composition comprising a composition having a blood pressure reducing and/or elevation suppressing effect of any one of items 1 to 9.

11. The composition of item 10, wherein said oral ingestible composition is a drink.

12. The composition of item 11, wherein said drink is a liquid coffee, an instant coffee or a coffee mixed drink.

13. The composition of item 10, wherein said oral ingestible composition is a food.

14. A method for treating high blood pressure in a human, said method comprising the human consuming foods and beverages containing an amount of mannose-based oligosaccharide mass, wherein the man nose-based oligosaccharide mass comprises 1 to 10 molecules of monosaccharides linked together, wherein the molecules of monosaccharides mainly comprise mannose, and wherein the amount contained in the food and drink is an effective amount to control the high blood pressure in the human.

15. A method for reducing the risk of developing high blood pressure in a human with normal blood pressure, said method comprising the human consuming foods and beverages containing an amount of mannose-based oligosaccharide mass, wherein the mannose-based oligosaccharide mass comprises 1 to 10 molecules of monosaccharides linked together, wherein the molecules of monosaccharides mainly comprise mannose, and wherein the amount contained in the food and drink is an effective amount to reduce the risk of developing high blood pressure in the human.

As a result of investigations of a blood pressure reducing and/or elevation suppressing effect, by the present inventors in order to solve the above described problem, with the use of the composition, obtained by the above described method, having a blood pressure reducing and/or elevation suppressing effect, and, comprising a mannose-based oligosaccharide mass comprising 1 to 10 molecules of monosaccharides linked together, said molecules mainly comprising mannose, it is found that a blood pressure reducing and/or elevation suppressing effect, is remarkably exhibited. Addition of a mannose-based oligosaccharide mass having a blood pressure reducing and/or elevation suppressing effect to food and drinks enables simple and economical ingestion in daily eating habits to expect the blood pressure reducing and/or elevation suppressing effect. The mannose-based oligosaccharide mass having a blood pressure reducing and/or elevation suppressing effect can be available from waste such as coffee extraction residues, and accordingly such resources that have not been used heretofore can also be effectively utilized.

The terms composition having, or a method providing or treating, “a blood pressure reducing and/or elevation suppressing effect” as used herein broadly means a composition or method having a function to generally reduce blood pressure, suppress elevation of blood pressure, prevent, reduce, treat, ameroliate, control, reduce the risk of, or otherwise effect high blood pressure or hypertension in a desirable manner. This term also encompasses, maintaining blood pressure in a normal range (especially for those at increased risk of developing high blood pressure), reducing the risk or delaying the development of high blood pressure or hypertension (especially for those with only moderately high blood pressure). This term also encompasses the use of the present composition in combination with other methods of controlling high blood pressure (e.g., other lifestyle changes, prescription blood pressure medication, and the like). Even in cases where the compositions of the present invention may not be able to fully control or maintain blood pressure in the desired range on their own, their use may allow the use of reduced levels of other blood pressure medications, thereby reducing the side effects of these other blood pressure medications.

### BRIEF DESCRIPTION OF DRAWING

FIG. 1 shows graphs of blood pressure change in humans consuming the mannooligosaccharide mass of this invention; FIG. 1A is for the systolic blood pressure and FIG. 1B is for the diastolic blood pressure. Data (n=24) used in this figure are expressed as averages ± standard deviation. In the figure, a single asterisk (*) indicates a significant difference compared to control group at p<0.05; a double asterisk (**) indicates a significant difference compared to control group at p<0.01; and a triple asterisk (***) indicates a significant difference compared to control group at p<0.001.

### DETAILED DESCRIPTION

In the present invention, the term “a mannose-based oligosaccharide mass” means a oligosaccharide mass comprising mannose (monosaccharide) as the main constituent element. The term “an oligosaccharide mass” as used herein generally means a substance which holds a position between a monosaccharide mass and a polysaccharide mass and is composed of a definite small amount of glycosyl bonds of monosaccharide molecules, in other words, a polymer having a comparatively small number of constituent monosaccharides. The term “an oligosaccharide mass” means a composition comprising a plurality of oligosaccharide molecules consisting of various numbers of constituent monosaccharides. And, the term “a mannose-based oligosaccharide mass” refers to a composition comprising a plurality of oligosaccharide molecules consisting of various types and numbers of constituent monosaccharides. In the present specification, the term “a mannooligosaccharide mass” is used in the same meaning as the term “a mannose-based oligosaccharide mass”.

In the present invention, the degree of polymerization of an oligosaccharide is sometimes described as “DP”. DP means the number of a monosaccharide constituting an oligosaccharide. In other words, mannose of a monosaccharide is represented by “DP 1”, and a mannooligosaccharide constituted of four mannose units has a degree of polymerization of four, namely, is represented by “DP 4”. From a technical viewpoint, the sugar having a degree of polymerization 1 (DP 1) is “a monosaccharide” and not an oligosaccharide. However, since an oligosaccharide mass (a mixture) used in the present invention sometimes contains monosaccharide molecules, even such oligosaccharide mass is generally called “a oligosaccharide mass” in the present specification. Namely, it should be understood that in the case of “a mannose-based oligosaccharide mass comprising 1 to 10 molecules of monosaccharides linked together”, this mixture of oligosaccharides sometimes contains monosaccharide molecules having a degree of polymerization of 1.

The mannose-based oligosaccharide mass used in the present invention is preferably a composition of a plurality of types of oligosaccharides comprising 1 to 10 molecules of monosaccharides linked together, said molecules comprising mainly mannose. Particularly preferred is a composition of oligosaccharides comprising 1 to 10 molecules of mannose linked together or a composition of a plurality of types of oligosaccharides comprising 1 to 10 molecules of monosaccharides linked together, said molecules comprising mannose and at least one monosaccharide selected from the group consisting of glucose, galactose, and fructose.

In the composition of the present invention, the total amount of the mannose-based oligosaccharide mass comprising 1 to 10 molecules of monosaccharides linked together is preferably not less than 60 percent, more preferably not less than 80 percent, based on the total solid content.

In the mannose-based oligosaccharide mass of the present invention, it is desired that the number of mannose residue is not less than 70 percent, preferably not less than 80 percent, of the total number of monosaccharide residues in the mannose-based oligosaccharide mass. Mannose-based oligosaccharide mass having levels of less than 70 percent of mannose residues based on the total number of residues generally do not significantly reduce blood pressure reducing and/or suppress elevation of blood pressure; moreover, levels of mannose residues below 70 percent based on the total number of residues increase sweetness, thereby making it more difficult to incorporate the present compositions in food or drink products without significantly effecting taste and other organoleptic properties. Thus, the level of mannose residues should be greater than 70 percent and the level of other residues less than 30 percent, based on the total number of residues. More preferably, the mannose resides should be greater than 80 percent and even more preferably greater than 90 percent with corresponding reduced levels of other residues, based on the total number of residues.

Further, it is preferred that the mannose-based oligosaccharide mass used in the present invention comprises 2 to 9 mannose units, and more preferably, 2 to 6 mannose units in the oligosaccharide molecules.

One embodiment of the present invention is a composition having a blood pressure reducing and/or elevation suppressing effect, and which comprises the above described mannose-based oligosaccharide mass. Thus, the composition having a blood pressure reducing and/or elevation suppressing effect according to the present invention can be produced with the use of the oligosaccharide having the above described blood pressure reducing and/or elevation suppressing effect.

Another embodiment of the present invention is a food and drink having an indication that this food and drink contains a composition having a blood pressure reducing and/or elevation suppressing effect characterized by having a human blood pressure reducing and/or elevation suppressing effect, and therefore being suited for eating and drinking by a person who is anxious about blood pressure.

The mannose-based oligosaccharide mass used in the present invention can be produced by hydrolyzing mannan. The raw material mannan herein can be obtained by extraction from, for example, coffee materials (especially spent coffee residues and other coffee-containing materials from commercial multistage coffee extraction systems), a copra meal or copra flake obtained from a coconut, Huacra Palm of a South African Arecaceae (Palmae) plant, a yam mannan, a Chinese yam mannan. Other mannan-containing plant species can also be used if desired. Generally mannan obtained from coffee materials, especially from spent coffee residues, are preferred.

A sugar mixture can be obtained by treating the mannan thus obtained by at least one method selected from acid hydrolysis, high temperature thermal hydrolysis, enzyme hydrolysis and microorganism fermentation, and preferably purifying the resulting hydrolysate by the method such as active carbon treatment, adsorptive resin treatment, ion-exchange resin treatment and ion-exchange membrane treatment. This mixture contains the above described mannose-based oligosaccharide mass having a blood pressure reducing and/or elevation suppressing function. The composition thus obtained has a blood pressure reducing and/or elevation suppressing function of the present invention. Furthermore, the composition having blood pressure reducing and/or elevation suppressing function of the present invention may be the one obtained by treating glucomannan present in a corn of konnyak, lily, narcissus, a lycoris and the like and galactomannan present in locust bean gum, guar gum and the like by at least one method selected from acid hydrolysis, high temperature thermal hydrolysis, enzyme hydrolysis and microorganism fermentation and then subjecting the resulting hydrolysate to purification treatment by the method such as active carbon treatment, adsorptive resin treatment, ion-exchange resin treatment and ion-exchange membrane treatment to increase the level of mannose as a constituent sugar.

Accordingly, mere “mannan” as used in the present specification broadly includes mannan of a polysaccharide having D-mannose alone as the constituent unit and, additionally, galctomannan and glucomannan of polysaccharides having mannose and galactose or glucoses as constituent units. Further, D-mannose is an aldohexose and an isomer of D-glucose differing only by having the opposite steric configuration of the hydroxyl group bonded to the carbon adjacent to the carboxyl group.

Furthermore, the composition having a blood pressure reducing and/or elevation suppressing effect of the present invention can be obtained by treating coffee raw beans or roasted coffee beans by at least one method selected from acid hydrolysis, high temperature thermal hydrolysis, enzyme hydrolysis and microorganism fermentation and purifying the resulting hydrolyzate by the method such as active carbon treatment, adsorptive resin treatment, ion-exchange resin treatment and ion-exchange membrane treatment. Or, the composition having a blood pressure reducing and/or elevation suppressing effect of the present invention may be obtained by treating spent coffee residues by at least one method selected from acid hydrolysis, high temperature thermal hydrolysis, enzyme hydrolysis and microorganism fermentation and purifying the resulting hydrolysate by the method such as active carbon treatment, adsorptive resin treatment, ion-exchange resin treatment and ion-exchange membrane treatment. In general, when roasted and ground coffee is extracted by a commercial extractor, at this instance, galactose of a side chain of a galactomannan present in the roasted coffee is solubilized and an arabinogalactan is solubilized by hydrolysis. Thus, it is presumed that mannan is rich in coffee residues and, in addition, mannan takes a straight-chain structure. On the other hand, cellulose is hard to decompose and remains in the residues but by suitably selecting conditions for specifically hydrolyzing mannan without decomposing cellulose, mannose-based oligosaccharide mass can be obtained.

Particularly, the methods of decomposing coffee extraction residues include a hydrolysis method with an acid and/or by high temperatures, a decomposition method with an enzyme and a decomposition method by microorganism fermentation and are not limited to them. The hydrolysis method with an acid and/or by high temperatures is disclosed in Japanese Patent Publication (A) Nos. Sho 61-96947 and Hei 02-200147 and the like. Spent coffee residues formed in the commercial multistage coffee extraction systems can be hydrolyzed in a reaction vessel with addition of an acid catalyst or can be hydrolyzed without addition of the acid catalyst after short time high temperature treatment. It is convenient to use a tubular plug flow reactor and use any reactor suited for conducting a short time comparatively high temperature reaction can give a good result. By controlling reaction time and reaction temperature, mannan having DP 10 to DP 40 is solubilized and hydrolyzed to decompose to oligosaccharide mass of DP 1 to DP 10, and then the hydrolyzates are separated from residues to obtain mannooligosaccharide mass. Further, coffee extraction residues as used herein mean so-called coffee extraction grounds after atmospheric or pressurized extraction of roasted and ground coffee with a solvent such as water.

When the composition having a blood pressure reducing and/or elevation suppressing effect is obtained by the hydrolysis treatment of coffee beans (including roasted beans and roasted and ground coffee beans), the type of coffee beans and their place of production are not limited, and any coffee beans of Coffea Arabica, Coffea Robusta, Coffea Liveria or the like may be used, and coffee beans produced in countries such as Brazil and Columbia can be used, and one type of beans alone or a blend of two or more types of beans may be used. Even coffee beans of inferior quality and/or size, which previously may have been rejected or disposed due to low commercial value, can be used. Roasted and ground coffee beans (including various forms of coarse-grinding, middle-grinding, middle fine-grinding, fine-grinding or the like using an ordinary grinder or a mill) which are obtained by roasting the above described beans very slightly, slightly, intermediately or deeply with the use of a generally employed roaster (of a hot air system, a far infrared system, a charcoal fire system or the like).

Further, coffee extraction residues which can be used may be any extraction residue obtained after the extraction treatment of roasted and ground coffee using a conventional process for producing liquid coffee or instant coffee through atmospheric or pressurized extraction and independently of the origin of the coffee beans and the preparation method.

Herein, the above described hydrolysis treatment will explained in detail. As the enzyme decomposition method, coffee extraction residues are suspended in an aqueous medium and, for example, a commercially available cellulase or hemicellulase may be added thereto and suspended while stirring. The amount of enzyme, the temperature and other conditions may be those which are employed in conventional enzymatic reactions without problems, and the optimum amount of action of the enzyme used, the temperature, and other conditions and factors may be suitably selected.

As the microorganism fermentation method, for example, microorganisms which produce cellulase, hemicellulase or the like may be planted in coffee extract residues suspended in an aqueous medium and cultured. The microorganisms used may be bacteria, Basidiomycota or the like which produce an enzyme capable of decomposing mannan in the coffee extraction residues, and cultivation conditions and the like may be suitably selected depending on the microorganisms used.

The reaction solution containing a composition having a blood pressure reducing and/or elevation suppressing effect comprising a mannose-based oligosaccharide mass can be purified, if necessary. As the purification method, the reaction solution is decolorized/deodorized by boneblack, active carbon, the carbonization method, adsorptive resins, the magnesia method, the solvent extraction method or the like, then desalted and deacidified by ion-exchange resins, ion-exchange membranes, electrodialysis or the like. A combination of purification methods and purification conditions may be suitably selected depending on the amounts of a pigment, a salt, an acid and the like present in the reaction solution comprising a mannose-based oligosaccharide mass and other factors.

Further, the present invention relates to an orally ingestible composition comprising the above explained composition having a blood pressure reducing and/or elevation suppressing action according to the present invention, particularly a food and drink. Furthermore, the composition having a blood pressure reducing and/or elevation suppressing effect, and according to the present invention can be used not only in the above described food and drink but also in the field of cosmetics, medicines and feed. The composition having a blood pressure reducing and/or elevation suppressing effect according to the present invention can be administered as the pharmaceutical composition having a blood pressure reducing and/or elevation suppressing effect as drugs or quasi-drugs. The composition according to the present invention may be administered as a pharmaceutical composition prepared by well known methods. Such pharmaceutical compositions includes tablets, powders, granules, fluids, syrups, and the like. The composition according to the present invention as drugs or quasi-drugs may be administered alone or may be mixed into food, drink, or feed. The composition having a blood pressure reducing and/or elevation suppressing effect, and according to the present invention exhibits the blood pressure reducing and/or elevation suppressing effect, and by oral ingestion, particularly as a food and drink. The intake or dose for obtaining the effect of the present invention is not limited and can be increased or decreased in response to body mass, age, the disease or symptoms of a person or a patient who ingest the composition. Generally, 0.1-40 g per day, preferably 0.5-20 g per day of the composition may be used for an adult.

For example, the composition having a blood pressure reducing and/or elevation suppressing effect, which is prepared so as to contain a high purified oligosaccharide mass by hydrolyzing coffee extract residues with an acid and/or by heating and which can be added to a liquid coffee, an instant coffee or the like as it is and used but for coffee richer in taste and flavor which coffee inherently possesses can be provided preferably by adding thereto a purified composition that has undergone a purification treatment such as decolorization, deodorization and deacidification with active carbon, ion-exchange resins, a solvent or the like as the need arises. Herein, liquid coffee includes a commercially available so-called coffee drink or coffee added drink which is put into a can or a so-called PET bottle container. Instant coffee includes so-called soluble powdered coffee obtained by removing water from an extract liquid obtained by extracting roasted and ground coffee with hot water by spraying or freeze-drying, and as a coffee mixed drink, for example, a drink obtained by adding sugar, a creaming powder and the like to soluble powdered coffee and mixed can be mentioned.

The composition having a blood pressure reducing and/or elevation suppressing effect, and the liquid coffee containing the same according to the present invention were prepared and the blood pressure reducing and/or elevation suppressing effect of this drink was examined as described in the following examples. Unless noted otherwise, all percentages are by weight. These examples specifically illustrates some embodiments of the present invention; they are not intended to limit the scope of the present invention.

### EXAMPLES

### Example 1

This Example illustrates the preparation of a mannose-based oligosaccharide mass suitable for use in the present invention. Roasted and ground coffee obtained by the conventional method was extracted by a commercially employed percolation system and the remaining coffee extraction residues were used. First, the coffee residues were ground to a particle diameter of about 1 mm to be easily fed into a reactor. Then, a slurry comprising water and ground materials and having a total solid content of about 14 percent was prepared, and heat-treated in a thermal plug flow reactor having a length of 4 m. The slurry thus obtained was pumped to a plug flow reactor with high-pressure steam at a speed corresponding to a residence time of eight minutes to be maintained at about 210° C. with the use of a 6.35 mm orifice. Thereafter, the resulting slurry was allowed to gush forth atmospherically to quickly stop the reaction. The resulting slurry was filtrated to separate a soluble solid content-containing liquid from an insoluble solid content. This soluble solid content-containing liquid was decolorized with active carbon and an adsorptive resin, and furthermore desalted with an ion-exchange resin, then concentrated and dried to obtain a composition comprising a mannose-based oligosaccharide mass comprising 1 to 10 molecules of monosaccharides linked together, said molecules mainly comprising mannose (14 percent total yield).

The DP distribution of oligosaccharide present in the thus obtained composition had DP 1 of 2.4 percent, DP 2 of 26.6 percent, DP 3 of 20.2 percent, DP 4 of 17.8, DP 5 of 10.9 percent, DP 6 of 8.9, and DP 7 of 6.0 percent, DP 8 of 3.6 percent, DP 9 of 1.9 percent, and DP 10 of 1.7 percent, and the content of a mannose residue in the sugar chain was 90 percent. The DP distribution and the content of the mannose residue in the sugar chain could be varied depending on the hydrolysis conditions employed. There were mannose and the like as oligosaccharides having DP 1; mannobiose and the like as oligosaccharides having DP 2; mannotriose and the like as oligosaccharides having DP 3; mannotetrose and the like as oligosaccharides having DP 4; mannopentose and the like as oligosaccharides of DP 5; mannohexose and the like as oligosaccharides of DP 6; mannoheptose and the like as oligosaccharides of DP 7; mannooctose and the like as oligosaccharides of DP 8; mannononose and the like as oligosaccharides of DP 9; and mannodecose and the like as oligosaccharides of DP 10. The bonding mode appears to be β-1,4-bonding.

### Example 2

This Example illustrates the effect of the mannose-based oligosaccharide mass obtained in Example 1 on the blood pressure in humans using a double blind clinical trial.

The subjects were twenty four males and twenty four females belonging in BMI 25 kg/m² group, and they were grouped into a control group (n=24) and a mannooligosaccharide ingestion group (n=24) so that the BMI of the two groups were about the same. The test drink was liquid coffee and the liquid coffee was prepared by diluting a concentrated coffee extract and artificial sweetener by water and then UHT sterilizing the diluted coffee. The obtained coffee was filled into a 900 mL PET bottle and the filled coffee was used as the control. The test drink for the mannooligosaccharide ingestion group was prepared by adding a liquid comprising mannooligosaccharide into liquid coffee (6 g of mannooligosaccharide per 300 mL). The subjects drank 300 mL test drink per day for twelve weeks, and the blood pressure of the subjects was checked at the start of the test, after four weeks, after eight weeks, and after twelve weeks.

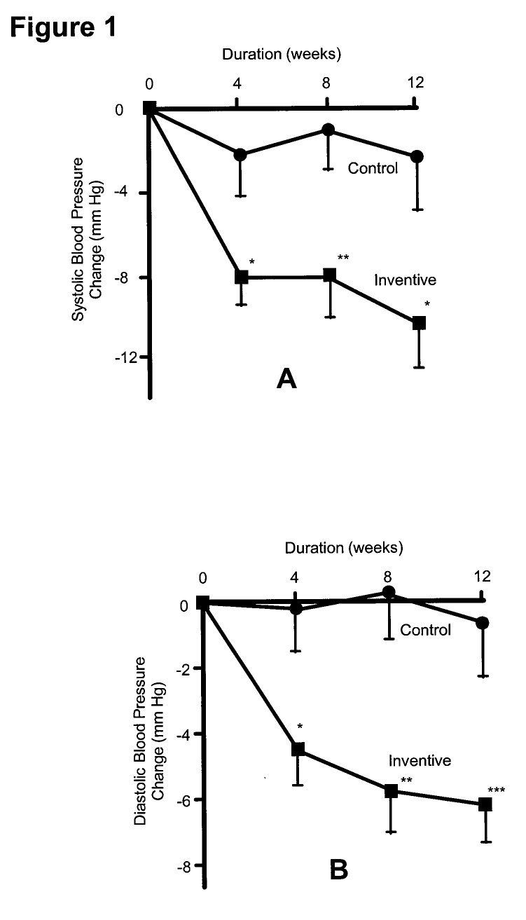

The blood pressure changes based on the start value are illustrated in FIG. 1. No significant blood pressure change was seen in the control group for twelve weeks. On the other hand, the significant blood pressure fall was seen in the mannooligosaccharide ingestion group demonstrating that ingestion of the drink comprising the mannooligosaccharide mass has a blood pressure reducing effect. Accordingly, the mannooligosaccharide mass has an effect of reducing blood pressure of human.

### Example 3

This Example illustrates the effect of the mannose-based oligosaccharide mass obtained in Example 1 on the blood pressure in rats. The rats used were susceptible to developing high blood pressure upon consuming salt (salt sensitive high blood pressure rat; Dahl S/Jr Sea). Rats were preliminarily raised for a week for inspection and conditioning and individuals that did not express any defect in the body mass transition and the general condition were used in the test. After the preliminary raising, rats were grouped into a control group consisting of five rats and a mannooligosaccharide ingestion group consisting of five rats so that the average body mass is about the same.

The feed was a mixture of powder feed for common feeding 700 g and cooking oil 300 g, and drinking water comprising 2 percent of salt was freely ingested from water feeding bottle and thereby high blood pressure was induced. Then purified mannooligosaccharide mass water solution was orally administered three times per day. The dose of mannooligosaccharide was 900 mg per day per individual. On the other hand, the same amount of purified water was orally administered to the control group. The blood pressure was checked at the start of the test, after two weeks, after 3 weeks and after four weeks.

The blood pressure changes based on the start value were shown in Table 1. By administering mannooligosaccharide mass, the significant effect of suppressing blood pressure elevation compared to the control group. Accordingly, administration of the mannooligosaccharide mass has an effect of suppressing blood pressure elevation of rat.

TABLE 1

| | Change (mm Hg) in Blood Pressure | | |
|---|---|---|---|
| | Relative to Initial Blood Pressure | | |
| Sample | 2 weeks | 3 weeks | 4 weeks |
| Control | +36.8 ± 5.0 | +22.2 ± 2.8 | +48.4 ± 4.9 |
| Inventive | +1.0 ± 8.33** | −5.6 ± 8.7* | +16.6 ± 3.2*** |

*Significant difference as compared to control group at p < 0.05.
**Significant difference as compared to control group at p < 0.01.
***Significant difference as compared to control group at p < 0.001.

1. A composition having a blood pressure reducing and/or elevation suppressing effect which comprises a mannose-based oligosaccharide mass comprising 1 to 10 residues of monosaccharides, wherein the residues of monosaccharides mainly comprise mannose.

2. The composition having the blood pressure reducing and/or elevation suppressing effect of claim 1, wherein the mannose-based oligosaccharide mass comprises 2 to 10 residues of mannose linked together.

3. The composition having a blood pressure reducing and/or elevation suppressing effect of claim 2, wherein the residues of monosaccharides comprise mannose and at least one monosaccharide selected from the group consisting of glucose, galactose, and fructose.

4. The composition having the blood pressure reducing and/or elevation suppressing effect of claim 2, wherein the mannose-based oligosaccharide mass comprises more than 60 percent based on total solid content of the composition.

5. The composition having the blood pressure reducing and/or elevation suppressing effect of claim 3, wherein the mannose-based oligosaccharide mass contains more than 70 percent mannose based on the total number of residues.

6. The composition having the blood pressure reducing and/or elevation suppressing effect of claim 5, wherein the mannose-based oligosaccharide mass contains 2 to 6 residues of mannose.

7. The composition having the blood pressure reducing and/or elevation suppressing effect of claim 1, wherein the mannose-based oligosaccharide mass is obtained by hydrolysis treatment of mannan.

8. The composition having the blood pressure reducing and/or elevation suppressing effect of claim 7, wherein the mannan is obtained from coffee beans, coffee extraction residues, or mixtures thereof.

9. The composition having the blood pressure reducing and/or elevation suppressing effect of claim 8, wherein the mannose-based oligosaccharide mass is β-1,4-mannooligosaccharide mass.

10. The composition having the blood pressure reducing and/or elevation suppressing effect of claim 8, wherein the composition is included in a food product suitable for consumption by a human.

11. The composition having the blood pressure reducing and/or elevation suppressing effect of claim 8, wherein the composition is included in a beverage suitable for consumption by a human.

12. The composition having the blood pressure reducing and/or elevation suppressing effect of claim 11, wherein the beverage is liquid coffee, instant coffee, or coffee-containing beverage.

13. A method for treating high blood pressure in a human, said method comprising the human consuming foods and beverages containing an amount of man nose-based oligosaccharide mass, wherein the mannose-based oligosaccharide mass comprises 2 to 10 residues of monosaccharides linked together, wherein the residues of monosaccharides mainly comprise mannose, and wherein the amount contained in the foods and the beverages is effective to provide a blood pressure reducing or elevation suppressing effect in the human when the foods and beverages are consumed by the human.

14. The method of claim 13, wherein the mannose-based oligosaccharide mass contains more than 70 percent mannose based on the total number of residues.

15. The method of claim 14, wherein the mannose-based oligosaccharide mass contains 2 to 6 residues of mannose.

16. The method of claim 15, wherein the mannose-based oligosaccharide mass is obtained by hydrolysis treatment of mannan.

17. The method of claim 16, wherein the mannan is obtained from coffee beans, coffee extraction residues, or mixtures thereof.

18. A method for reducing the risk of developing high blood pressure in a human with normal blood pressure, said method comprising the human consuming foods and beverages containing an amount of mannose-based oligosaccharide mass, wherein the mannose-based oligosaccharide mass comprises 2 to 10 resides of monosaccharides linked together, wherein the residues of monosaccharides mainly comprise mannose, and wherein the amount contained in the food and drink is effective to reduce the risk of developing high blood pressure in the human when the foods and beverages are consumed by the human.
